(19) United States
(12) Patent Application Publication
(10) Pub. No.: US 2012/0190007 A1
(43) Pub. Date: Jul. 26, 2012

(54) METHOD AND SYSTEM FOR MONITORING AND RECORDING A VIRAL INFECTION PROCESS AND THAT FOR SCREENING VACCINES

(75) Inventors: CHIH-WEI WU, KEELUNG CITY (TW); CHANG-JER WU, TAIPEI CITY (TW); SHIH-HAO HUANG, KAOHSIUNG CITY (TW); SHENG-PING CHANG, NEW TAIPEI CITY (TW)

(73) Assignee: NATIONAL TAIWAN OCEAN UNIVERSITY, KEELUNG CITY (TW)

(21) Appl. No.: 13/185,996

(22) Filed: Jul. 19, 2011

(30) Foreign Application Priority Data
Jan. 20, 2011 (TW) 100102179

(51) Int. Cl. C12Q 1/70; C12M 1/34

(52) U.S. Cl. 435/5; 435/287.1; 435/288.7

(57) ABSTRACT

The present invention relates to a method and system for monitoring and recording a viral infection process and that for screening vaccines, which is characterized by providing a microcantilever detection device, which comprises a microcantilever comprising a contact area having an macromolecular material attached thereon; allowing host cell to be attached to the macromolecular material; allowing a sample containing a test virus or vaccine to be loaded into the contact area to make the test virus or vaccine to contact the host cells attached to the macromolecular material so as to produce a deflection of the microcantilever; and recording the deflection in a time course manner so as to obtain a deflection profile that can used as a basis for determining the viral infection process or the potential efficacy of the vaccines.

### CROSS-REFERENCE TO RELATED APPLICATION PARAGRAPH

This application claims the benefit of Taiwan Patent Application No. 100102179 filed on Jan. 20, 2011, the content of which is hereby incorporated by reference in its entirety.

### TECHNOLOGY FIELD

The present invention relates to a method and system for monitoring and recording a viral infection process in host cells and that for screening vaccines, drug, and health food.

### BACKGROUND OF THE INVENTION

In general, a viral infection process in host cells comprises three phases: (1) the penetration phase: targeting and entering the host cells; (2) the replication phase: replicating nucleic acids and proteins needed for the virus to live; (3) the transmission phase: leaving the infected cell and further infecting other cells. A thorough understanding of the viral infection process not only provides a basis for developing anti-virus drugs or vaccines, but also is helpful for providing accurate timing for administering medicine, so as to treat diseases effectively. However, most of the current virus detection technologies, including reverse transcriptase-polymerase chain reaction (RT-PCR), virus isolation, enzyme-linked immunosorbent assay (ELISA), polymerase chain reaction (PCR) and the like, as well as microcantilever biological sensor technology that is being developed recently, are to detect the presence of a virus based on specific binding interaction between certain molecules, such as specific interaction between primers, probe or antibodies and antigens, but none of those can be used to monitor and record a complete viral infection process in host cells.

In addition, vaccine is one of the main approaches to prevent viral infection. However, the current vaccine screening technologies are mainly based on cell or animal experiments, which are time-consuming and inefficient.

### BRIEF SUMMARY OF THE INVENTION

The present invention relates to a detection technology, which can be used for monitoring and recording a viral infection process in host cells and that for screening effective vaccines, characterized by providing a microcantilever detection device, which comprises a microcantilever comprising a contact area having an macromolecular material attached thereon; allowing host cell to be attached to the macromolecular material; allowing a sample containing a test virus or vaccine to be loaded into the contact area to make the test virus or vaccine to contact the host cells attached to the macromolecular material so as to produce a deflection of the microcantilever; and recording the deflection in a time course manner so as to obtain a deflection profile that can used as a basis for determining the viral infection process or the potential efficacy of the vaccines.

Therefore, in one aspect, the present invention provides a method for monitoring and recording an infection process of a test virus in host cells, comprising:

(a) providing a microcantilever detection device, which comprises a microcantilever comprising a contact area having a macromolecular material attached thereon; wherein the macromolecular material is hydrophilic and biocompatible;

(b) loading the host cells to the contact area to allow the host cells to be attached to the macromolecular material;

(c) conducting a detection, which comprises loading a sample containing the test virus to the contact area, where the host cells are attached to the macromolecular material, to allow the test virus to contact and infect the host cells, and therefore to produce a deflection of the microcantilever; and measuring the deflection in a time course manner so as to give a deflection profile; and

(e) monitoring and recording the infection process of the test virus in the host cells based on the deflection profile.

In another aspect, the present invention provides a method for determining a potential efficacy of a test vaccine to protect host cells from infection of a test virus, comprising:

(a) providing a microcantilever detection device, which comprises a microcantilever comprising a contact area having a macromolecular material attached thereon; wherein the macromolecular material is hydrophilic and biocompatible;

(b) loading the host cells to the contact area to allow the host cells to be attached to the macromolecular material;

(c) conducting a first detection, which comprises loading a sample containing the test virus to the contact area, where the host cells are attached to the macromolecular material, to allow the test virus to contact and infect the host cells, and therefore to produce a first deflection of the microcantilever; and measuring the first deflection in a time course manner so as to give a first deflection profile having a first slope;

(d) conducting a second detection, which comprises loading a sample containing the test virus and the test vaccine to the contact area, where the host cells are attached to the macromolecular material, to allow the test vaccine to interact with the test virus or the host cells, and therefore to produce a second deflection of the microcantilever; and measuring the second deflection in a time course manner so as to give a second deflection profile having a second slope;

(e) comparing the first slope of the first deflection profile and the second slope of the second deflection profile, and determining, based on the comparison, the potential efficacy of the test vaccine to protect the host cells from infection of the test virus, wherein the first slope of the first deflection profile smaller than the second slope of the second deflection profile indicates that the test vaccine is of said potential efficacy.

In a further aspect, the present invention provides a system for monitoring and recording an infection process of a test virus in host cells, comprising:

(a) a microcantilever detection device, which comprises a microcantilever comprising a contact area having a hydrophilic and biocompatible macromolecular material attached thereon for fixing the host cells and a signal detecting area; wherein the microcantilever can produce a deflection when the test virus is loaded to the contact area, where the host cells are attached to the macromolecular material, and therefore contacts and infects the host cells;

(b) a signal detecting device, comprising a signal producing means for producing a detectable signal responsible to the deflection and a signal receiving means for receiving the detectable signal and converting it to an outputting signal; and

(c) a signal processing device for receiving the outputting signal and converting it to a recordable data so as to give a deflection profile in a period of time of measurement which is used for monitoring and recording the infection process of the test virus in the host cells.

It is believed that a person of ordinary knowledge in the art where the present invention belongs can utilize the present invention to its broadest scope based on the descriptions herein with no need of further illustration. Therefore, the following descriptions should be understood as of demonstrative purpose instead of limitative in any way to the scope of the present invention.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows an embodiment according to the microcantilever detection device of the present invention.

FIG. 2 shows (a) a specific embodiment according to the microcantilever detection device of the present invention (π-shaped microcantilever); and (b) the fabrication process of the microcantilever and PDMS microfluidics in one embodiment of the invention; and (c) use of the gelatin gel as a sacrificial layer to solve the sticking problem between the PDMS microfluidics and the hydrogel microstructure.

FIG. 3 shows a diagram of the detection technology for monitoring and recording the viral infection process in host cells in one embodiment of the present invention, wherein [symbol] means the flow direction, [symbol] means a macromolecular material (e.g. hydrogel), [symbol] means UV light, [symbol] means host cells, [symbol] means a test virus, (a) means that a macromolecular material (e.g. hydrogel) solution is added to the microcantilever; (b) means UV light exposure; (c) means that the macromolecular material (e.g. hydrogel) is cured into a solid, the host cells are loaded to the macromolecular material, and the microcantilever generates deflection h1; (d) means that a sample containing the test virus is loaded to the macromolecular material (e.g. hydrogel); (e) means that the test virus is attached to the host cells in the macromolecular material (e.g. hydrogel), and the microcantilever generates deflection h2; (f) means that the virus enters and replicates in the host cells, and the microcantilever generates deflection h3; and (g) means that virus leaves the host cells, and the microcantilever deflection goes back to h0.

FIG. 4 shows an embodiment according to the microcantilever detection device of the present invention 101, which comprises a microcantilever 2, a hydrogel material 6 attached to the contact area thereof, PDMS microfluidics 14, a silicon wafer substrate 16, a microfluidic inlet 141 and a microfluidic outlet 142.

FIG. 5 shows an embodiment according to the detection system of the present invention, comprising a He—Ne laser source 102, a space filter 104, a pinhole 105, focus lens 106, refractive lens 108, a position sensing detector 110, a microcantilever detection device 101, a first reflected beam 118, a second reflected beam 120, and a signal processing device 112.

FIG. 6 is a photograph showing that the microfluidic system chip was bond to the microcantilever chip as described in Example 1.

FIG. 7 is a diagram showing the relationship between the light signal and the displacement of the microcantilever, wherein the laser beam 109 is focused on the optical detection area of the microcantilever 2, and the beam is reflected to the four-quadrant position sensing detector 110; and the displacement d of the reflected light is measured, from which the deflection ΔZ of the microcantilever detection device is calculated, wherein d means the displacement of the reflected laser beam on the four-quadrant position sensing detector; h means the distance between microcantilever detection device and the four-quadrant position sensing detector; and L means the length of the microcantilever detection device.

FIG. 8 shows a specific example of an optical detection system according to the present invention, including a He—Ne laser source 102, a space filter 104, focus lens 106, refractive lens 108, a position sensing detector 110, a microcantilever detection device 101, a charge-coupled device (CCD) 114, and a sample stage 116.

FIG. 9 shows the deflection profile of the microcantilever in one embodiment of the invention obtained as in Example 3.

FIG. 10 shows the deflection profile of the microcantilever in one embodiment of the invention obtained as in Example 3.

FIG. 11 shows the situations where the host cells were attached to the hydrogel material and infected by the viruses as in example 3, which were simultaneously observed with a fluorescence microscope of the optical detection system according to the present invention. Scale: 500 μm.

FIG. 12 shows the deflection profile of the microcantilever according to the present invention detected in example 3.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The present invention relates to a method and system for monitoring and recording a viral infection process in host cells and that for screening vaccines by using a microcantilever detection technology.

In one aspect, the present invention provides a method for monitoring and recording an infection process of a test virus in host cells, comprising:

(a) providing a microcantilever detection device, which comprises a microcantilever comprising a contact area having a macromolecular material attached thereon; wherein the macromolecular material is hydrophilic and biocompatible;

(b) loading the host cells to the contact area to allow the host cells to be attached to the macromolecular material;

(c) conducting a detection, which comprises loading a sample containing the test virus to the contact area, where the host cells are attached to the macromolecular material, to allow the test virus to contact and infect the host cells, and therefore to produce a deflection of the microcantilever; and measuring the deflection in a time course manner so as to give a deflection profile; and

(e) monitoring and recording the infection process of the test virus in the host cells based on the deflection profile.

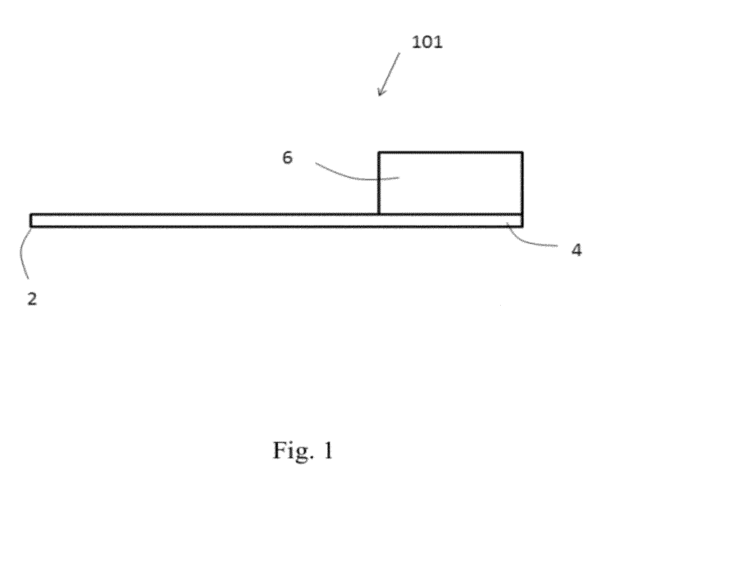

As shown in FIG. 1, in an embodiment according to the present invention, a microcantilever detection device 101 comprises a microcantilever 2, wherein the microcantilever comprises a contact area 4, on which a macromolecular material 6 is attached.

As used herein, the term “macromolecular material” is a hydrophilic and biocompatible macromolecular material, which can be used to fix cells and is non-toxic to the cells. In one embodiment, the macromolecular material is a hydrogel material.

As used herein, the term “hydrogel material” is a water-absorbing gel. Hydrogel material is hydrophilic and biocompatible, which can absorb water to swell while keeping its three dimensional (3D) structure. There are various hydrogel materials that can be used herein, including but are not limited to, polyhydroxyethylmethacrylate (PHEMA) hydrogel, polyethylene glycol diacrylate (PEGDA) hydrogel, gelatin methacrylate (GelMA) hydrogel, alginate hydrogel, alginate hydrogel, chitosan hydrogel and agarose hydrogel and the like. In order to allow a hydrogel material to be fixed on microcantilever, typically, a crosslinking agent and a photoinitiator are added to a hydrogel material solution, to allow the hydrogel material solution to be used in a micromolding process, and the hydrogel material is cured into a solid in the contact area of the microcantilever by using an exposure method.

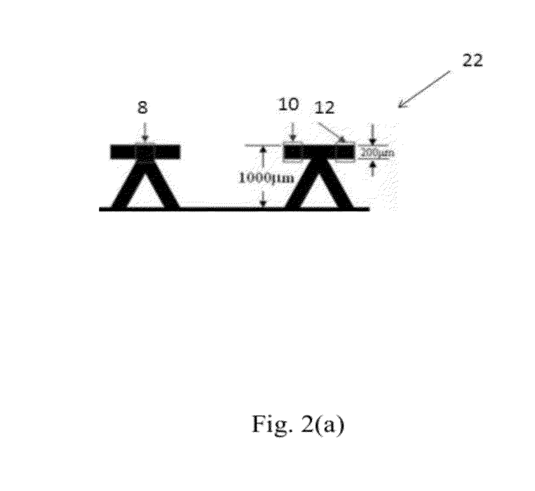

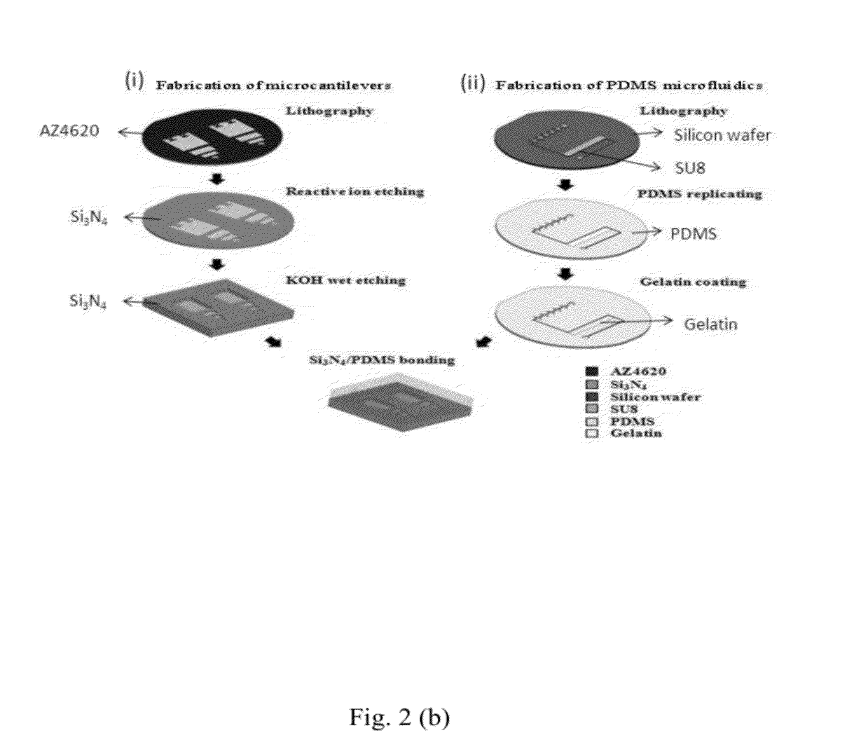

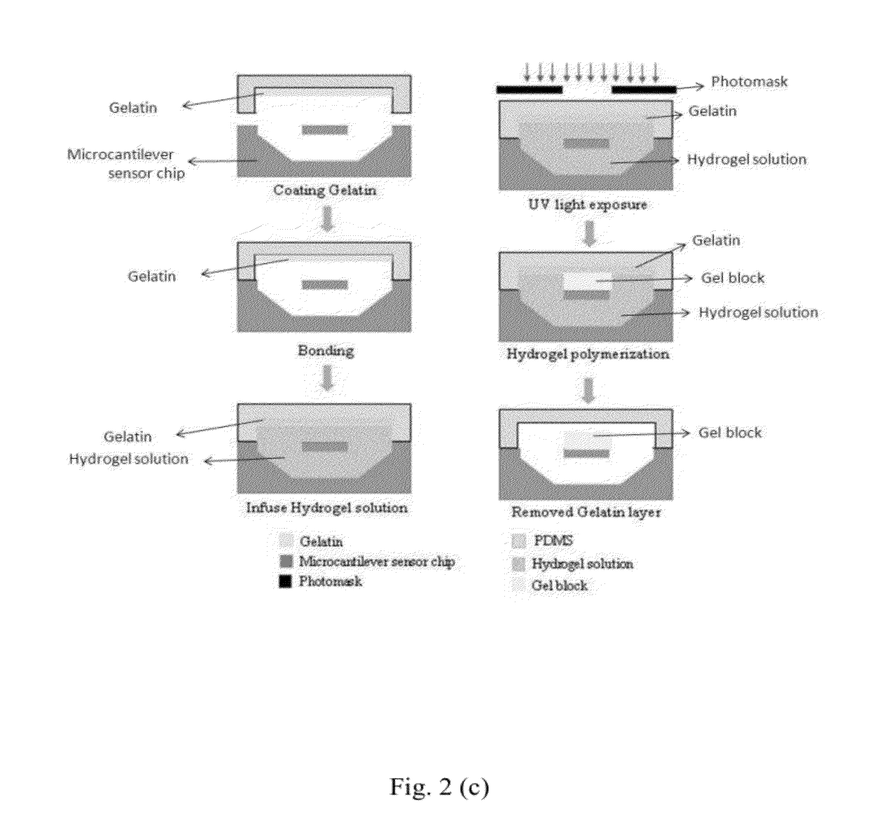

As used herein, the term “microcantilever” may be a microcantilever sensor generally used in this art, which produces a small deformation in structure with a slight force (e.g. deflection), and a detection with high sensitivity can be conducted based on such small deformation. In one embodiment, the microcantilever is it-shaped, which has good resistance to torsion force. As shown in FIG. 2(a), in a certain embodiment, the microcantilever of the present invention is a π-shaped microcantilever 22, having a hydrogel material as the macromolecular material for fixing host cells, wherein the middle part of the it-shaped microcantilever 22 is designed as a hydrogel material exposing area 8, the size of which is about 200 μm×200 μm that is designed based on the line width of the microcantilever; both ends of the π-shaped microcantilever 10, 12 may be used as optical detection areas for the incident and reflected laser beam, to avoid light shining directly on the uneven hydrogel material, or to avoid refraction which may cause consequential difficulty for a position sensing detector to receive a light signal and subsequently inaccuracy of signal measurement, and also prevent cells from damages caused by the high energy laser beam.

It is demonstrated based on the synchronous observation via fluorescence microscope that, the deflection profile of the microcantilever obtained according to the present invention can represent a viral infection process in host cells. Typically, a viral infection process in host cells includes a penetration phase, a replication phase and a transmission phase. In one embodiment of the invention, when the deflection of the microcantilever reaches a maximum value in the deflection profile, it means the timing that the replication phase ends and transmission phase is going to start.

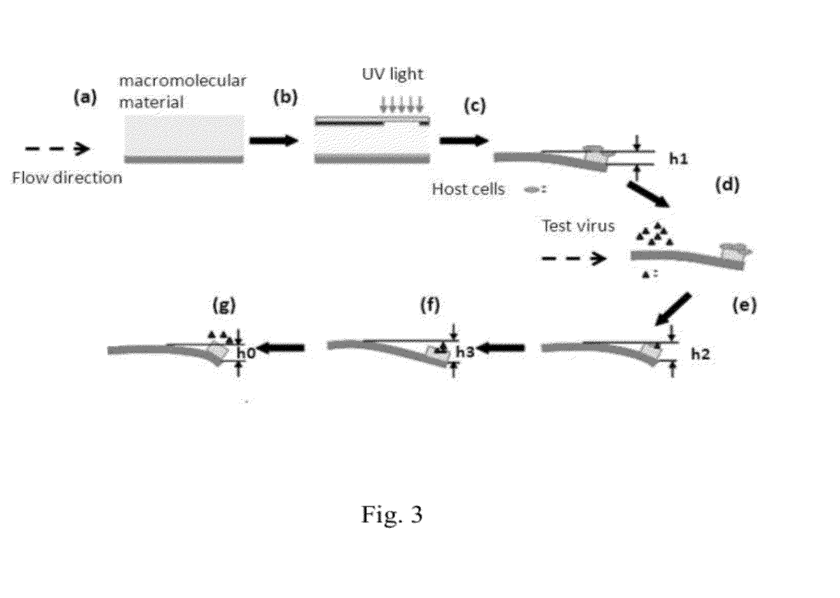

As shown in FIG. 3, in one embodiment of the present invention, a hydrogel material is used as the macromolecular material for fixing host cells. Specifically, a hydrogel material solution is cured to a solid on the microcantilever through UV irradiation, and then a sample containing host cells is loaded to the microcantilever so that the host cells are attached to the hydrogel material; at this time, the microcantilever produces a deflection h1. Subsequently, a sample containing a test virus is loaded to the microcantilever to allow the test virus infects the host cells. During the infection process while the virus contacts with the cells (penetration phase), replicates in the cells (replication phase), and leaves the cells (transmission phase), microcantilever generates deflections h2, h3 and h0 over time, wherein h2 represents the penetration phase, h3 represents the replication phase, and h0 represents the phase that the virus leaves the host cells, wherein h3>h2>h1, and when h0<h3, it means the deflection reaches a maximum value and then the microcantilever goes back to its original position.

The deflection generated by the microcantilever may be detected by a common detection methods known in this art, including but are not limited to, an optical detection approach, an acoustic detection approach, an electric detection approach, or a magnetic detection approach.

In one embodiment, the microcantilever detection device of the present invention may further comprises a microfluidic system, through which a sample containing the test virus is loaded to the contact area of the microcantilever to allow the test virus to contact and infect the host cells fixed thereon.

Typically, the microcantilever detection device of the present invention is fabricated using a four-inch silicon wafer. The fabrication procedures of the microcantilever begins with a low stress silicon nitride layer deposited on a four-inch silicon wafer followed by a photolithographic process using a photoresist, which defines the pattern of the microcantilever, and a reactive ion etching system is then used to selectively etch away the undefined photoresist portion of low stress silicon nitride by SF6 and O2 gases. The uncovered portion of the substrate was etched by potassium hydroxide solution to release the microcantilevers. On the other hand, the fabrication procedures of a polydimethylsiloxane (PDMS) microfluidic system typically begin with a silicon wafer coated with a photoresist, to create a mold master, including an inlet, an outlet, several bubble traps, and a chamber for cells adhered and cultured on the microcantilever. Following soft baking of the photoresist, the photomask patterns are transferred to the photoresist coated silicon wafer. Post-exposure bake, development, and hard bake of the exposed photoresist patterns are followed by pouring the mixture of PDMS onto the patterns. The master is then replicated with PDMS as shown in FIG. 2(b). A thin gelatin film used as a sacrificial layer is then deposited onto the bottom of the cell laden chamber of the PDMS replica to create a space for cells to be laden and cultured and to avoid the hydrogel microstructure sticking to the PDMS replica when hydrogel was exposed to UV light from the top side of the PDMS replica (see FIG. 2(b)). A hydrogel material solution is prepared and loaded to the microcantilever through the microfluidic system and cured into a solid on the microcantilever, and subsequently the remaining hydrogel solution is washed away and the sacrificial layer (a gelatin film) is removed. Finally, the surface modified PDMS cast is bonded onto the silicon wafer containing a microcantilever by using oxygen plasma bonding technology, and the microcantilever detection device of the present invention is accomplished.

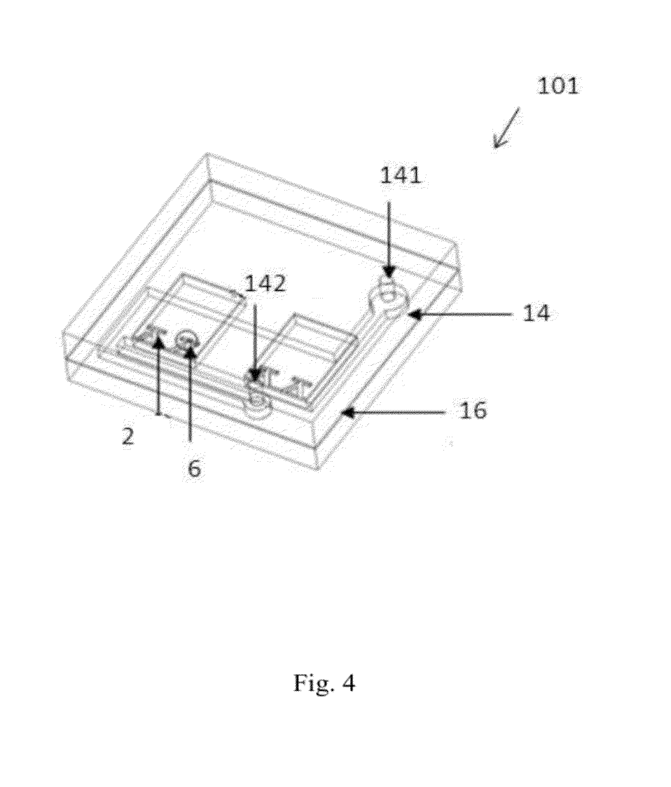

In one embodiment, as shown in FIG. 4, the microcantilever detection device of the present invention 101 comprises a microcantilever 2, a hydrogel material 6 attached on the contact area of the microcantilever, a PDMS microfluidic system 14, a silicon wafer substrate 16, an inlet of the microfluidic system 141 and an outlet of the microfluidic system 142.

In addition, the microcantilever detection device of the present invention can further be used for screening vaccines. In this connection, after loading a sample containing the host cells to allow the host cells to be attached to the macromolecular material, a separate detection is conducted, including loading a sample containing a test virus and a test vaccine, wherein if the test vaccine has a potential efficacy to protect host cells from viral infection, it is expected that the deflection of the microcantilever as measured would tend to stop increasing over time, i.e. the slope of the deflection profile tends to be steady.

Therefore, in another aspect, the present invention provides a method for determining a potential efficacy of a test vaccine to protect host cells from infection of a test virus, comprising:

(a) providing a microcantilever detection device, which comprises a microcantilever comprising a contact area having a macromolecular material attached thereon; wherein the macromolecular material is hydrophilic and biocompatible;

(b) loading the host cells to the contact area to allow the host cells to be attached to the macromolecular material;

(c) conducting a first detection, which comprises loading a sample containing the test virus to the contact area, where the host cells are attached to the macromolecular material, to allow the test virus to contact and infect the host cells, and therefore to produce a first deflection of the microcantilever; and measuring the first deflection in a time course manner so as to give a first deflection profile having a first slope;

(d) conducting a second detection, which comprises loading a sample containing the test virus and the test vaccine to the contact area, where the host cells are attached to the macromolecular material, to allow the test vaccine to interact with the test virus or the host cells, and therefore to produce a second deflection of the microcantilever; and measuring the second deflection in a time course manner so as to give a second deflection profile having a second slope;

(e) comparing the first slope of the first deflection profile and the second slope of the second deflection profile, and determining, based on the comparison, the potential efficacy of the test vaccine to protect the host cells from infection of the test virus, wherein the second slope of the second deflection profile smaller than the first slope of the first deflection profile indicates that the test vaccine is of said potential efficacy.

In addition, in a further aspect, the present invention provides a system for monitoring and recording an infection process of a test virus in host cells, comprising:

(a) a microcantilever detection device, which comprises a microcantilever comprising a contact area having a hydrophilic and biocompatible macromolecular material attached thereon for fixing the host cells and a signal detecting area; wherein the microcantilever can produce a deflection when the test virus is loaded to the contact area, where the host cells are attached to the macromolecular material, and therefore contacts and infects the host cells;

(b) a signal detecting device, comprising a signal producing means for producing a detectable signal responsible to the deflection and a signal receiving means for receiving the detectable signal and converting it to an outputting signal; and

(c) a signal processing device for receiving the outputting signal and converting it to a data so as to give a deflection profile in a period of time of measurement which is used for monitoring and recording the infection process of the test virus in the host cells.

According to the present invention, the signal detecting device may be established based on optical detection approach, an acoustic detection approach, an electric detection approach, or a magnetic detection approach. In one embodiment, the signal detecting device is establish based on an optical detecting approach (i.e. an optical detecting device), which comprises a laser source, a spatial filter, a focusing lens set, refractive lens, a position sensing detector, wherein the laser source provides a beam of laser light that goes through the space filter to form an uniform beam, which then goes through the focusing lens set to form a parallel beam, which further goes through the refractive lens to form a first reflected beam 118, which is subsequently focused on the signal detecting area of said microcantilever detection device and forms a second reflected beam 120, and the position sensing detector receives the second reflected beam and converting it to an electrical outputting signal.

In a certain example, the system of the invention further comprises a charge coupled device for observing whether the first reflected beam is focused on the signal detecting area of said microcantilever detection device. In another example, said charge coupled device may also be used for observing the host cells, for example, to confirm the different phases of the infection process.

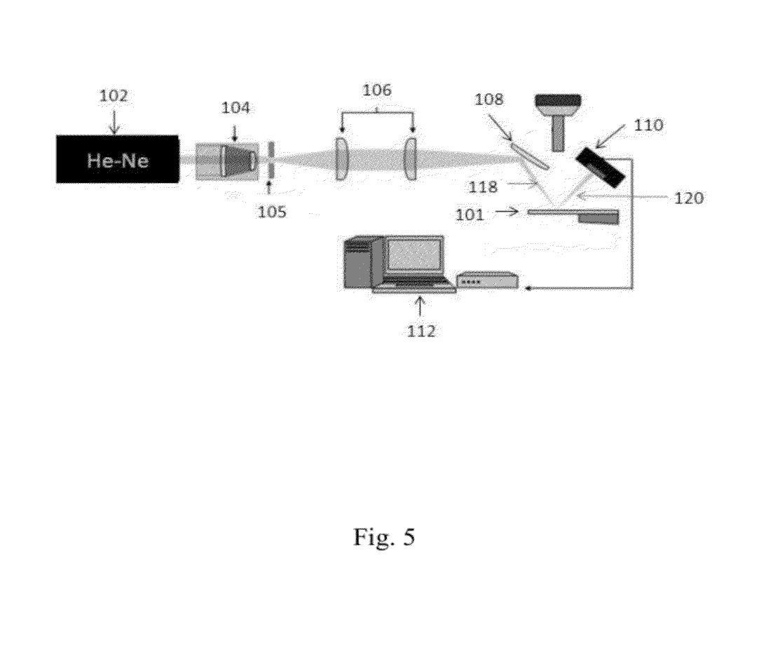

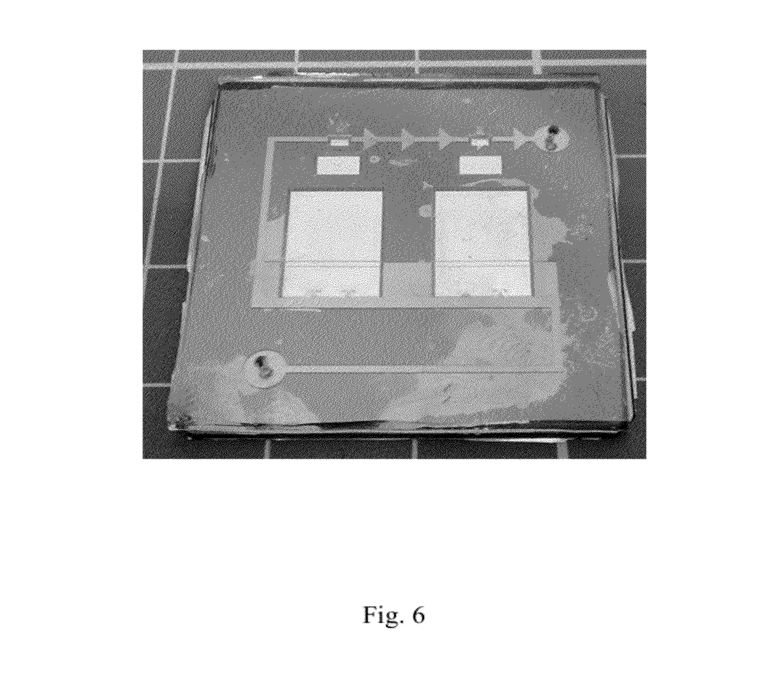

As shown in FIG. 5, in one embodiment, the system of the present invention is an optical detection system, comprising a He—Ne laser source 102, a space filter 104, a pinhole 105, a focusing lens set 106, refractive lens 108, a position sensing detector 110, a microcantilever detection device 101, a first reflected beam 118, a second reflected beam 120, and a signal processing device 112.

In a specific example, the present invention provides a system for monitoring and recording the infection process of a test virus in host cells, comprising:

(a) a microcantilever detection device, which comprises a microcantilever comprising a contact area having a hydrophilic and biocompatible macromolecular material attached thereon for fixing the host cells and an optical signal detecting area; wherein the microcantilever can produce a deflection when the test virus is loaded to the contact area, where the host cells are attached to the macromolecular material, and therefore contacts and infects the host cells;

(b) an optical detecting device, comprising a laser source, a spatial filter, a focusing lens set, refractive lens, a position sensing detector, wherein the laser source provides a beam of laser light that goes through the space filter to form an uniform beam, which then goes through the focusing lens set to form a parallel beam, which further goes through the refractive lens to form a first reflected beam, which is subsequently focused on the signal detecting area of said microcantilever detection device and forms a second reflected beam, and the position sensing detector receives the second reflected beam and converts it to an electrical outputting signal; and

(c) a signal processing device for receiving the electrical outputting signal and converting it to a data representing said deflection so as to give a deflection profile in a period of time of measurement which is used for monitoring and recording the infection process of the test virus in the host cells.

The system of the invention can also be used for screening drugs (e.g. an antibiotic drug) or healthy food.

The various embodiments of the present invention are described in details below. The technical characteristics of the present invention will be more clearly presented by the following detailed descriptions about the various embodiments and claims.

### EXAMPLE

### Example 1

### Design and Fabrication of the Microcantilever Detection Device of the Invention

FIG. 2(b) shows the fabrication procedure of a microcantilever biochip consisting of a silicon nitride microcantilever and PDMS microfluidics. Bulk micromachined microcantilevers were fabricated using commercial four-inch silicon wafers, which were first cleaned using standard Piranha solution and RCA cleaning processes. The fabrication procedures of microcantilevers began with a 1 μm low stress silicon nitride thin layer being deposited on a silicon wafer followed by photolithographic processes using AZ4620 photoresist and photomask-1, which defined the pattern of the π-shape microcantilevers by aligner (AB-M InC., U.S.A.). The whole area of the π-shaped microcantilever was 1000 μm in length and 1000 μm in width. The length and width of the front-bar of the π-shaped microcantilevers were 1000 μm and 200 μm, respectively. The spring constant of the microcantilever was only 0.0328 N/m, which was less than most commercially available silicon nitride cantilevers with spring constants of 0.12-0.05 N/m. The PHEMA hydrogel microstructure for laden cells must be deposited on the center of the front-bar to increase the sensitivity of the microcantilever. The laser spot light used to measure the microcantilever deflections could be located on the sides of the front-bar to avoid damaging cells adhering to the PHEMA hydrogel. Moreover, the reflection location of the laser beam will be uncertain during the period of microcantilever deflection if the laser spot light is guided onto the 3D PHEMA microstructure and cells. The silicon nitride thin film was used as an anisotropic masking layer, and reactive ion etching (Crie-100, Advanced System Technology, Taiwan) was then followed to selectively etch away the masking layer using SF6 and O₂ gases. The uncovered portion of the silicon substrate was etched by potassium hydroxide solution to release the microcantilevers. These microcantilevers were then rinsed in acetone followed by methanol, and deionized water (DI water) as shown in FIG. 2(b).

The fabrication procedures of PDMS microfluidics began with a silicon wafer coated with SU-8 photoresist to create a mold master, including an inlet, an outlet, several bubble traps, and a chamber for cells adhered and cultured on microcantilevers as shown in FIG. 2(b). Following soft baking of SU-8 photoresist, the photomask-2 patterns were transferred to the SU-8 coated silicon wafer by the above-mentioned aligner. Post-exposure bake, development, and hard bake of the exposed SU-8 patterns were followed by pouring the mixture of PDMS onto the patterns. The prepolymer was mixed with its curing agents in a 10:1 ratio, and was degassed in a vacuum chamber by an aspirator (AS-3, Newlab Instruments, UK) for at least 30 min at room temperature before pouring the mixture onto the patterns. The master was then replicated with PDMS for several minutes at 90° C. as shown in FIG. 2(b). However, the surface of the PDMS replica is hydrophobic; thus, its surface must be changed to have a hydrophilic property before coating with gelatin and bonding onto a silicon substrate. In this work, the PDMS replica was rinsed in ethanol and then subjected to oxygen plasma using the above-mentioned reactive ion etching apparatus Crie-100 for 50 sec at a power of 20 watts and an oxygen flow rate of 50 sccm. A thin gelatin film used as a sacrificial layer was then deposited onto the bottom of the cell laden chamber of the PDMS replica to create a space for cells to be laden and cultured and to avoid the hydrogel microstructure sticking to the PDMS replica when hydrogel was exposed to UV light from the top side of the PDMS replica as shown in FIG. 2(b). Finally, the surface modified PDMS cast was bonded onto the micromachined silicon wafer containing microcantilevers as shown in FIG. 2(b).

Photolithography was utilized to micropattern PHEMA onto microcantilevers. After injecting the PHEMA hydrogel solution into the inlet of the microcantilever-based biochip, a photomask-3 was used to selectively expose the PHEMA solution by UV light with an intensity of 10 mW/cm2 for 120 seconds. Afterward, 40° C. deionized water was injected into the biochip to remove the uncrosslinked PHEMA solution and the gelatin layer, releasing the microcantilever. The biochips containing microcantilevers with PHEMA microstructures were then injected with DI water and exposed to UV light several times to completely remove the residual DMSO solvent in PHEMA microstructures and disinfect the whole chip. The size of the hydrogel microstructure on the n-shaped microcantilever was about 200 μm×200 μm.

### Example 2

### Design and Establishment of the Optical Detection System

An optical detection system having the microcantilever detection device as described was designed and established for monitoring and recording a viral infection process in host cells.

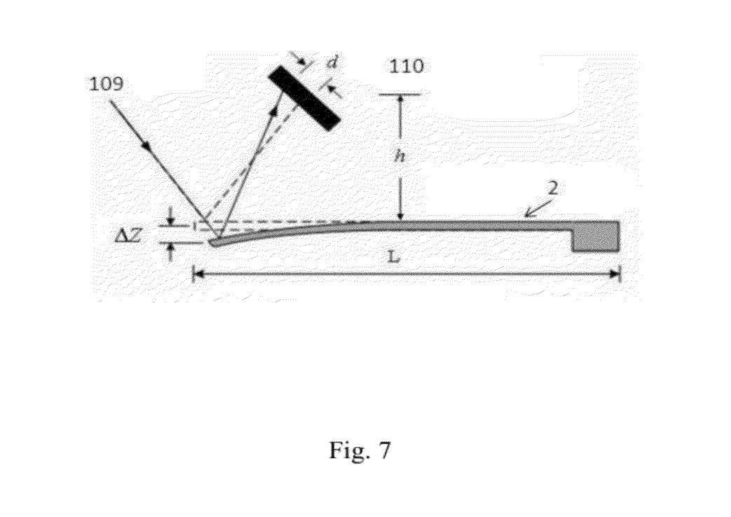

A low-power He—Ne laser was used as light source. The intensity of the laser light was equalized and the luminous flux was decreased via space filter and pinhole. The route of the light beam with defined size was refracted by a refractive lens, and then the light beam focused on the optical detection area of the microcantilever, and the beam reflected to the four-quadrant position sensing detector (Position-Sensitive detector, PSD) because of the optical lever principle. The displacement d of the reflected light was measured by the four-quadrant position sensing detector, from which the deflection of the microcantilever detection device was calculated, as shown in FIG. 7.

The deflection ΔZ of the microcantilever detection device may be calculated based on the optical lever principle and triangle geometry:

$$Δ Z = \frac{d}{h} L$$

d: the displacement of the reflected laser beam on the four-quadrant position sensing detector

h: the distance between microcantilever detection device and the four-quadrant position sensing detector

L: the length of the microcantilever detection device

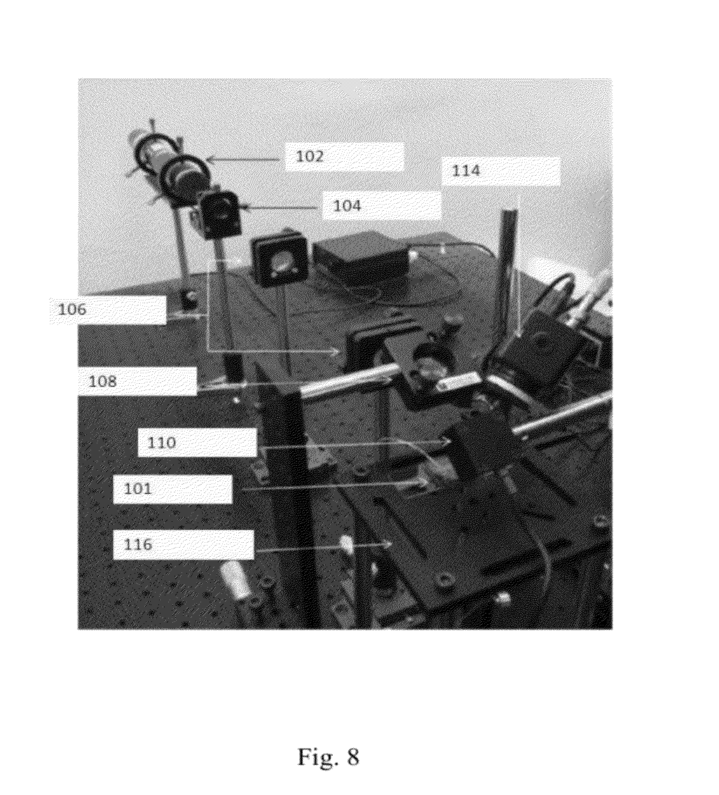

In the optical detection system of the present invention, a charge-coupled device (CCD) was also used to observe whether the laser beam was focused accurately on the optical detection area. The signal received by the four-quadrant position sensing detector was amplified by an amplifier and recorded in a computer. In addition, in the optical detection system of the present invention, fluorescence microscope CCD lens may also be used to observe synchronously the growing of cells on the hydrogel material, in order to further understand the complete viral infection process and compare it with the result of traditional viral infection detection methods. The optical detection system of the present invention is shown in FIG. 8.

### Example 3

### Monitoring and Recording a Viral Infection Process in Host Cells

3.1 Cell Preparation

BHK-21 cells (Baby hamster kidney cell line, ATCC CCL-10) were used in this example. Cells were cultured in the medium containing trypsin and Ethylenediaminetetraacetic acid (EDTA) at 37° C., so that cells could adhere to the culture plate and grow.

3.2 Loading Cell Medium and Detecting the Deflection of the Microcantilever

BHK-21 cells were seeded on the PHEMA hydrogel microstructure at a concentration of 1.6×10⁶ cells/ml in 1 ml medium. First, all the 1 ml medium contained BHK-21 cells was injected into the microcantilever-based biochip at 100 μl/min flow rate by a syringe pump (Harvard Apparatus, USA), then the pump was turned off. The volume of the chamber was around 290 μl. The chamber was in static condition after injecting to allow BHK-21 cells to adhere naturally onto the hydrogel microstructure. A living and dead assay were then performed with ethidium homodimer and calcein AM. These two stains were added to PBS, and then injected into the chamber of the microcantilever based biochip to stain BHK-21 cells laden on the PHEMA hydrogel microstructure. After rinsing with PBS, cells were evaluated by a fluorescence microscopy (BX51, Olympus, Japan). According to the cell fluorescent signals, it was found that cells adhered to hydrogel material surface and most of them are healthily alive (data not shown).

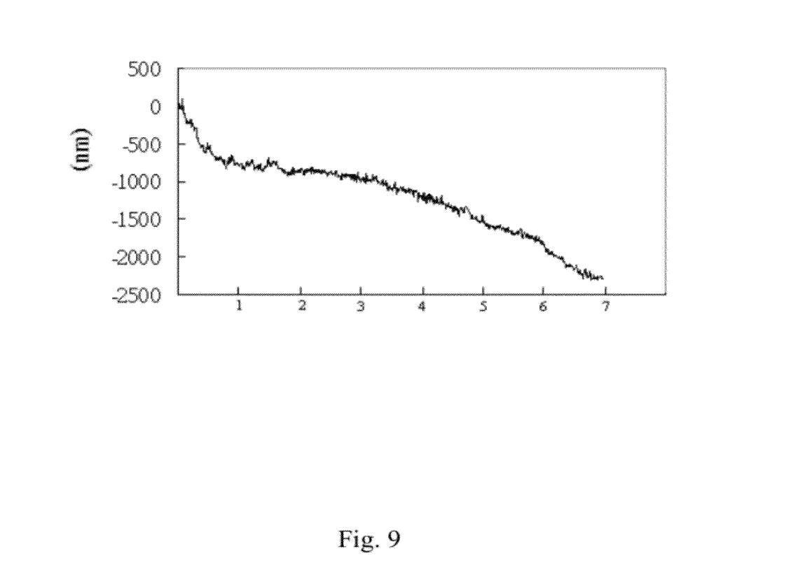

Meanwhile, deflections of the microcantilever were measured by the optical detection system as shown in FIG. 8, including a He—Ne laser source 102, a space filter 104, focus lens 106, refractive lens 108, a position sensing detector 110, a microcantilever detection device (chip) 101, a charge-coupled device (CCD) 114, and a sample stage 116. FIG. 9 shows that the optical detection system of the present invention successfully detected the signals in responsive to the deflection generated by the microcantilever, wherein the deflection increased continually during the cell-laden period.

3.3 Loading a Virus Sample and Monitoring and Recording the Infection Process in Host Cells

After the host cells were attached to the hydrogel microstructure for 7 hours, a phosphate buffer containing Japanese encephalitis virus was loaded into the microfluidic system, to make the virus contact the cells of the hydrogel material of the microcantilever detection device. Viral infection in cells was expressed by M.O.I. (Multiplicity Of Infection), which was the ratio of the amount of virus and the amount of infected cells. The M.O.I value in this study was 1, namely one virus infecting one cell. The cycle of Japanese encephalitis virus including entering to the cells, replicating in the cells and leaving the cells was about 6 hour. In this study, signals responsive to deflection of the microcantilever detection device were collected and recorded in a 7-hour period of time after the cells were infected by the virus.

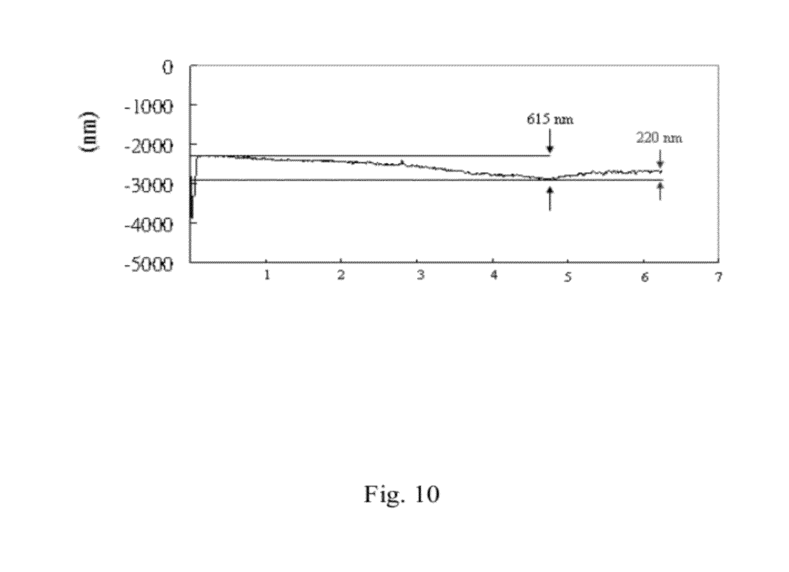

As shown in FIG. 10, after the phosphate buffer containing Japanese encephalitis virus was loaded and the cells were infected by the virus, the microcantilever started to deflect downwards, which implied that the virus was continuously infecting the cells and started to replicate in the cells. In addition, the deflection of the microcantilever later increased to a level of 615 nm at 4 hours and 40 minutes after cells were infected by the virus; and after this moment, the deflection level started to decrease, which implied that the virus has completed the replication and started to leave the cells, and further during the subsequent 2-hour period of time, the deflection of the microcantilever decreased to a level of 220 nm, which implied that the replication was completed and the virus continuously left the cells.

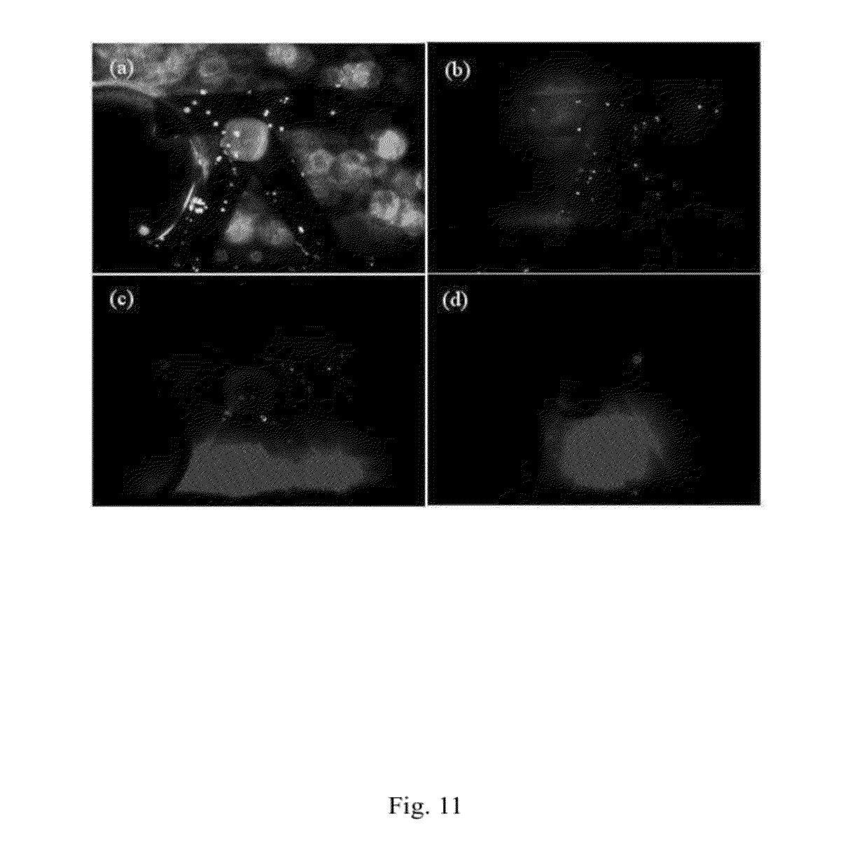

Qualitative observations of the cells were conducted with fluorescence microscope after the cells were infected at 4 hours and 7 hours. As shown in FIG. 11, in the control group, BHK-21 cells attached to the hydrogel material for 11 hours without being infected by Japanese encephalitis virus, were mostly healthily alive (a); in the experimental groups, after BHK-21 cells were attached to the hydrogel material for 7 hours, virus was loaded to the cells, and observations were made when the cells were infected by the virus at 4 hours (b) and 7 hours (c), from which it was found that the cells gradually died because of the viral infection and detached from the surface of the hydrogel material, reflecting the result of the decrease of the microcantilever deflection; and after cells were infected at 7 hours, the microfluidic system was washed with phosphate butter, and it was observed that almost all the cells died and detached from the surface of the hydrogel material (d).

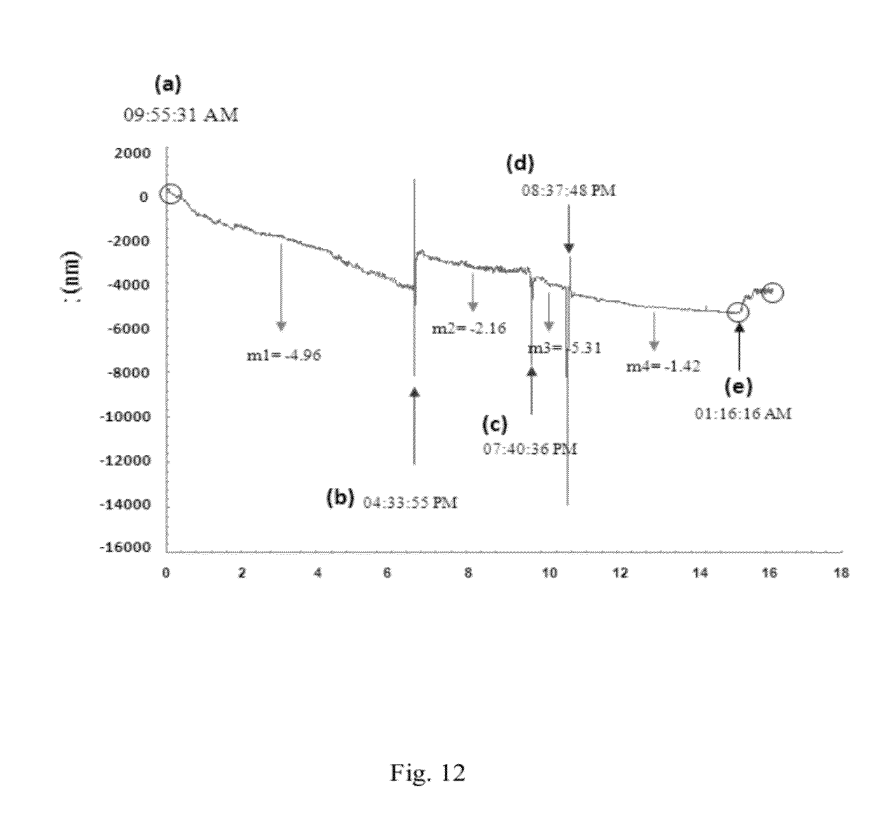

Independently, a deflection profile of the microcantilever was measured and recorded by using BHK-21 cells (2.4×10⁶ cells/ml) and Japanese encephalitis virus (M.O.I: 1), for a period of 16 hours and 15 minutes. As shown in FIG. 12, at the time point (a) being 09:55:31 AM when the cells were loaded and during a period of time being about 6 hours and 38 minutes (the cells being attached to the hydrogel material), the slope of the deflection profile was ml (−4.96); at the time point (b) being 04:33:55 PM, 5 ml cell medium was used to wash away the cells that were not attached to the hydrogel material, and when the system went stable, the slope of the deflection profile was m2 (−2.16), which was used to make sure that the following deflection was resulted from the subsequent viral infection, instead of the unwanted suspending cells; at the time point (c) being 07:40:30 PM, a sample containing the virus was loaded, the slope of the deflection profile was m3 (−5.31); subsequently at the time point (d) being 08:37:48 PM, 5 ml cell medium was used to wash away the virus that did not enter the cells, which was used to make sure that the following deflection was resulted from the subsequent viral replication, instead of the unwanted suspending virus, and when the system went stable, the slope of the deflection profile was m4 (−5.31), indicating that the virus was entering the cells and replicated to lead to increased deflection; and finally at the time point (e) being 01:16:14 AM, the microcantilever deflected upwards, indicating that the replicated virus left the cells.

In addition, in order to screen vaccines, an independent experiment could be conducted by loading a sample containing the virus and a test vaccine after the cell attachment, and obtaining a deflection profile of the microcantilever during the same period of time. It would be expected that if the test vaccine can protect cells from infection, the deflection would not increase, namely the slope of the deflection profile would remain the same, or smaller than said slope m3.

The above results show that, the technical scheme proposed by the present invention can be used for monitoring and recording viral infection process in host cells, as well as for screening vaccines.

It is believed that a person of ordinary knowledge in the art where the present invention belongs can utilize the present invention to its broadest scope based on the descriptions herein with no need of further illustration. Therefore, the following descriptions should be understood as of demonstrative purpose instead of limitative in any way to the scope of the present invention. The publications referred to herein are incorporated by reference in their entireties.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method for monitoring and recording an infection process of a test virus in host cells, comprising:
(a) providing a microcantilever detection device, which comprises a microcantilever comprising a contact area having a macromolecular material attached thereon; wherein the macromolecular material is hydrophilic and biocompatible;
(b) loading the host cells to the contact area to allow the host cells to be attached to the macromolecular material;
(c) conducting a detection, which comprises loading a sample containing the test virus to the contact area, where the host cells are attached to the macromolecular material, to allow the test virus to contact and infect the host cells, and therefore to produce a deflection of the microcantilever; and measuring the deflection in a time course manner so as to give a deflection profile; and
(e) monitoring and recording the infection process of the test virus in the host cells based on the deflection profile.

2. The method of claim 1, wherein the macromolecular material is a hydrogel material.

3. The method of claim 2, wherein the hydrogel material is selected from the group consisting of polyhydroxyethylmethacrylate (PHEMA) hydrogel, polyethylene glycol diacrylate (PEGDA) hydrogel, gelatin methacrylate (GelMA) hydrogel, alginate hydrogel, alginate hydrogel, chitosan hydrogel and agarose hydrogel.

4. The method of claim 1, wherein the microcantilever is π-shaped.

5. The method of claim 1, wherein the infection process comprises a penetration phase, a replication phase and a transmission phase.

6. The method of claim 5, wherein in the deflection profile, a maximum level of the deflection is indicative of ending of the replication phase and starting of the transmission phase.

7. The method of claim 1, wherein the deflection is measured by an optical detection approach, an acoustic detection approach, an electric detection approach, or a magnetic detection approach.

8. The method of claim 1, wherein the microcantilever detection device further comprises a microfluidic system, through which the sample containing the test virus is loaded to the contact area of the microcantilever to allow the test virus to contact and infect the host cells fixed thereon.

9. A method for determining a potential efficacy of a test vaccine to protect host cells from infection of a test virus, comprising:
(a) providing a microcantilever detection device, which comprises a microcantilever comprising a contact area having a macromolecular material attached thereon; wherein the macromolecular material is hydrophilic and biocompatible;
(b) loading the host cells to the contact area to allow the host cells to be attached to the macromolecular material;
(c) conducting a first detection, which comprises loading a sample containing the test virus to the contact area, where the host cells are attached to the macromolecular material, to allow the test virus to contact and infect the host cells, and therefore to produce a first deflection of the microcantilever; and measuring the first deflection in a time course manner so as to give a first deflection profile having a first slope;
(d) conducting a second detection, which comprises loading a sample containing the test virus and the test vaccine to the contact area, where the host cells are attached to the macromolecular material, to allow the test vaccine to interact with the test virus or the host cells, and therefore to produce a second deflection of the microcantilever; and measuring the second deflection in a time course manner so as to give a second deflection profile having a second slope; and
(e) comparing the first slope of the first deflection profile and the second slope of the second deflection profile, and determining, based on the comparison, the potential efficacy of the test vaccine to protect the host cells from infection of the test virus, wherein the second slope of the second deflection profile smaller than the first slope of the first deflection profile indicates that the test vaccine is of said potential efficacy.

10. A system for monitoring and recording an infection process of a test virus in host cells, comprising:
(a) a microcantilever detection device, which comprises a microcantilever comprising a contact area having a hydrophilic and biocompatible macromolecular material attached thereon for fixing the host cells and a signal detecting area; wherein the microcantilever can produce a deflection when the test virus is loaded to the contact area, where the host cells are attached to the macromolecular material, and therefore contacts and infects the host cells;
(b) a signal detecting device, comprising a signal producing means for producing a detectable signal responsible to the deflection and a signal receiving means for receiving the detectable signal and converting it to an outputting signal; and
(c) a signal processing device for receiving the outputting signal and converting it to a data so as to give a deflection profile in a period of time of measurement which is used for monitoring and recording the infection process of the test virus in the host cells.

11. The system of claim 10, wherein the signal detecting device is established based on an optical detecting approach, an acoustic detecting approach, an electric detecting approach, or a magnetic detecting approach.

12. The system of claim 10, wherein the signal detecting device is establish based on an optical detecting approach, which comprises a laser source, a spatial filter, a focusing lens set, refractive lens, a position sensing detector, wherein the laser source provides a beam of laser light that goes through the spatial filter to form an uniform beam, which then goes through the focusing lens set to form a parallel beam, which further goes through the refractive lens to form a first reflected beam, which is subsequently focused on the signal detecting area of said microcantilever detection device and forms a second reflected beam, and the position sensing detector receives the second reflected beam and converting it to an electrical outputting signal.

13. A system for monitoring and recording the infection process of a test virus in host cells, comprising:
(a) a microcantilever detection device, which comprises a microcantilever comprising a contact area having a hydrophilic and biocompatible macromolecular material attached thereon for fixing the host cells and an optical signal detecting area; wherein the microcantilever can produce a deflection when the test virus is loaded to the contact area, where the host cells are attached to the macromolecular material, and therefore contacts and infects the host cells;
(b) an optical detecting device, comprising a laser source, a spatial filter, a focusing lens set, refractive lens, a position sensing detector, wherein the laser source provides a beam of laser light that goes through the spatial filter to form an uniform beam, which then goes through the focusing lens set to form a parallel beam, which further goes through the refractive lens to form a first reflected beam, which is subsequently focused on the signal detecting area of said microcantilever detection device and forms a second reflected beam, and the position sensing detector receives the second reflected beam and converts it to an electrical outputting signal; and
(c) a signal processing device for receiving the electrical outputting signal and converting it to a data representing said deflection so as to give a deflection profile in a period of time of measurement which is used for monitoring and recording the infection process of the test virus in the host cells.

14. The system of claim 10, wherein the macromolecular material is a hydrogel material.

15. The system of claim 14, wherein the hydrogel material is selected from the group consisting of polyhydroxyethylmethacrylate (PHEMA) hydrogel, polyethylene glycol diacrylate (PEGDA) hydrogel, gelatin methacrylate (GelMA) hydrogel, alginate hydrogel, alginate hydrogel, and chitosan hydrogel.

16. The system of claim 10, wherein the microcantilever is 7r-shaped.

17. The system of claim 12, wherein the system further comprises a charge coupled device for observing whether the first reflected beam is focused on the signal detecting area of said microcantilever detection device.

18. The system of claim 13, wherein the system further comprises a charge coupled device for observing whether the first reflected beam is focused on the signal detecting area of said microcantilever detection device.

19. The system of claim 17, wherein the charge coupled device is also used for observing the host cells during the infection process.

20. The system of claim 18, wherein the charge coupled device is also used for observing the host cells during the infection process.
